(19) United States
(12) Patent Application Publication
(10) Pub. No.: US 2017/0326252 A1
(43) Pub. Date: Nov. 16, 2017

(54) Nanoparticles for Delivery of Agents to Glioblastoma Tumors

(71) Applicant: Nanoforma Inc., Oakland, CA (US)

(72) Inventor: Amy Shuen, Oakland, CA (US)

(73) Assignee: Nanoforma Inc., Oakland, CA (US)

(21) Appl. No.: 15/587,407

(22) Filed: May 5, 2017

Related U.S. Application Data
(60) Provisional application No. 62/334,283, filed on May 10, 2016.

(51) Int. Cl. A61K 47/69; C12N 7/00; A61K 51/12; A61K 47/64; A61K 31/704

(52) U.S. Cl. CPC A61K 47/6929; A61K 31/704; A61K 51/1203; A61K 51/1244; A61K 47/64; C12N 7/00; C12N 2770/00032; C12N 2770/00023

(57) ABSTRACT

The present invention is, in general, directed to nanoparticles for the delivery of agents to glioblastoma tumors. More particularly, the present invention relates to nanoparticle conjugates that deliver and release agents to a glioblastoma tumor. The invention is also directed to methods of delivering agents to glioblastoma tumors.

This application claims the benefit of provisional application Ser. No. 62/334,283 filed on May 10, 2016.

### FIELD OF THE INVENTION

The present invention is, in general, directed to nanoparticles for the delivery of agents to glioblastoma tumors. More particularly, the present invention relates to nanoparticle conjugates that deliver and release agents to a glioblastoma tumor. The invention is also directed to methods of delivering agents to glioblastoma tumors.

### BACKGROUND OF THE INVENTION

Glioblastomas (GBM) are tumors that arise from astrocytes—the star-shaped cells that make up the “glue-like,” or supportive tissue of the brain. These tumors are usually highly malignant (cancerous) because the cells reproduce quickly and are supported by a large network of blood vessels. Glioblastomas are generally found in the cerebral hemispheres of the brain, but can be found anywhere in the brain or spinal cord.

Glioblastoma represents about 15% of all primary brain tumors and about 60-75% of all astrocytomas. They increase in frequency with age, and affect more men than women. Glioblastoma can be difficult to treat because the tumors contain so many different types of cells. Some cells may respond well to certain therapies, while others may not be affected at all. This is why the treatment plan for glioblastoma may combine several approaches. Other complicating factors in the treatment of glioblastoma include, for example, glioblastoma tumor cells are often resistant to conventional therapies, brain tissue surrounding glioblastomas are sensitive to conventional therapies, the brain has a limited capacity to repair damage from conventional therapies, many drugs have difficulty crossing the blood-brain barrier, and glioblastomas have finger-like tentacles that make it difficult to completely remove.

With standard treatment, median survival for adults with an anaplastic astrocytoma is about two to three years. For adults with more aggressive glioblastoma, treated with concurrent temozolamide and radiation therapy, median survival is about 14.6 months and two-year survival is 30%. However, a 2009 study reported that almost 10% of patients with glioblastoma may live five years or longer.

It is an object of the invention to provide nanoparticles that reach and accumulate in glioblastoma tumors. It is also an object of the invention to deliver agents to a glioblastoma tumor using the nanoparticles of the invention.

### SUMMARY OF THE INVENTION

The present invention provides nanoparticles that reach and accumulate in glioblastoma tumors. In some embodiments, the nanoparticles are viral nanoparticles made from viral proteins. In some embodiments, the nanoparticles are made from bacteriophage proteins. In some embodiments, the nanoparticles are cylindrical in shape. In some embodiments, the nanoparticle cylinders have a diameter of 5 to 20 nanometers, and a length of 10 to 150 nanometers. In some embodiments, the nanoparticle cylinders have a diameter of 5 to 20 nanometers, and a length of 10 to 100 nanometers. In some embodiments, the nanoparticle cylinders have a diameter of 5 to 10 nanometers, and a length of 25 to 50 nanometers. In some embodiments, the nanoparticles are disk shaped. In some embodiments, the nanoparticle disks have a diameter of 5-40 nanometers and a thickness of about 5 to 25 nanometers. In some embodiments, the nanoparticle disks have a diameter of 10-20 nanometers and a thickness of about 5 to 15 nanometers. In some embodiments, viral nanoparticles are engineered using phage display methodologies to include an antibody binding portion, a polypeptide signal for attachment of a moiety (e.g., biotin), or amino acids for conjugation to a linker or directly to the agent.

In some embodiments, the nanoparticles are conjugated to agents to make nanoparticle agent conjugates. In some embodiments, the nanoparticles are conjugated to reporters. In some embodiments, the nanoparticles are conjugated to imaging agents. In some embodiments, the nanoparticles are conjugated to therapeutics. In some embodiments, the nanoparticles are conjugated to chemotherapeutic agents. In some embodiments, the nanoparticles are conjugated to one or more of a mitotic inhibitor, an antitumor antibiotic, an immunomodulating agent, a gene therapy agent, an alkylating agent, an antiangiogenic agent, an antimetabolite, a chemoprotective agent, an antihormone agent, a corticosteroid, a photoactive therapeutic agent, an oligonucleotide, a radionuclide, a topoisomerase inhibitor, a tyrosine kinase inhibitor, or another agent. In some embodiments, the nanoparticles are conjugated to a mitotic inhibitor, an antitumor antibiotic, a plant alkaloid, an alkylating agent, an antimetabolite, or a radionuclide. In some embodiments, the nanoparticles are conjugated to a mitotic inhibitor. In some embodiments, the nanoparticles are conjugated to a plant alkaloid. In some embodiments, the nanoparticles are conjugated to an antitumor antibiotic. In some embodiments, the nanoparticles are conjugated to an alkylating agent. In some embodiments, the nanoparticles are conjugated to an antimetabolite. In some embodiments, the nanoparticles are conjugated to a radionuclide.

In some embodiments, a linker is used to conjugate the agent and the nanoparticle together. In some embodiments, the linker is multifunctional. In some embodiments, the linker has a reactive group for attaching to the nanoparticle and a reactive group for attaching to the agent. In some embodiments, the linker is cleavable. In some embodiments, the linker is noncleavable. In some embodiments, the cleavable linker is stable outside of the glioblastoma tumor and the linker is cleaved at the tumor. In some embodiments, the linker is stable extracellularly and cleaved when internalized into a tumor cell. In some embodiments, the linker is cleavable by a protease. In some embodiments, the linker is self-immolative. In some embodiments, the linker releases the agent in response to a change in pH. In some embodiments, the linker includes a hydrophilic polymer such as, for example, PEG or a polysaccharide. In some embodiments, the linker includes one or more of 6-maleimidocaproyl, maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl)cyclohexane-1 carboxylate (“MCC”).

In some embodiments, the linker has the formula NR—AA—AR where NR is a group that can attach to the nanoparticle and can optionally include an additional spacer molecule, AA is an optional amino acid unit that can include a peptide cleavable by a protease, and AR is a group that can attach to the agent and can optionally include an additional spacer molecule. The AR spacer may also include a self-immolative group for release of the agent from the conjugate. In some embodiments, the linker and/or NR group attaches to the nanoparticle at a delta amino group of lysine residues in the nanoparticle, or at a thiol group of cysteine residues in the nanoparticle.

In some embodiments, the nanoparticles of the invention also include an antibody moiety. In some embodiments, the antibody moiety is included in a viral nanoparticle using phage display approaches to include the antibody moiety in a viral protein that is part of the nanoparticle. In some embodiments, an antibody moiety is conjugated to the nanoparticle using chemistries and linkers described above. In some embodiments, the antibody moiety binds to CD15, CD133, A2B5, integrin α6, podoplanin, and/or other surface marker found on glioblastoma tumor cells. In some embodiments, the antibody moiety is a single chain antibody, or a recombinant antibody fragment, or a protease cleavage fragment of an antibody (Fab or F(ab)₂).

In some embodiments, the nanoparticles and/or nanoparticle agent conjugates have a half-life of 1, 2, 3, 4, 5, 6, 7, 8, 9, 10, 11,or 12 hours, or 1, 2, 3, 4, 5, 6, or 7 days, or 2, 3, or 4 weeks, or 2, 3, 4, 5, or 6 months. In some embodiments, the nanoparticle agent conjugates have a stability half-life outside of the glioblastoma tumor of 1, 2, 3, 4, 5, 6, 7, 8, 9, 10, 11,or 12 hours, or 1, 2, 3, 4, 5, 6, or 7 days, or 2, 3, or 4 weeks, or 2, 3, 4, 5, or 6 months.

In some embodiments, the nanoparticles of the invention are used to transport an agent to a glioblastoma tumor. In some embodiments, the nanoparticles of the invention transport a reporter to the glioblastoma tumor. In some embodiments, the nanoparticles transport a reporter that allows the glioblastoma tumor to be imaged. In some embodiments, the nanoparticles of the invention transport to the glioblastoma tumor a mitotic inhibitor, an antitumor antibiotic, an immunomodulating agent, a gene therapy agent, an alkylating agent, an antiangiogenic agent, an antimetabolite, a chemoprotective agent, an antihormone agent, a corticosteroid, a photoactive therapeutic agent, an oligonucleotide, a radionuclide, a topoisomerase inhibitor, a tyrosine kinase inhibitor, or another agent. In some embodiments, the nanoparticles of the invention transport a mitotic inhibitor to a glioblastoma tumor. In some embodiments, the nanoparticles of the invention transport an antitumor antibiotic. In some embodiments, the nanoparticles of the invention transport an alkylating agent to the glioblastoma tumor. In some embodiments, the nanoparticles of the invention transport an antimetabolite to a glioblastoma tumor.

In some embodiments, the nanoparticles of the invention are used to transport agents to and accumulate agents in a glioblastoma tumor. In some embodiments, the nanoparticles of the invention accumulate in a glioblastoma tumor a mitotic inhibitor, an antitumor antibiotic, an immunomodulating agent, a gene therapy agent, an alkylating agent, an antiangiogenic agent, an antimetabolite, a chemoprotective agent, an antihormone agent, a corticosteroid, a photoactive therapeutic agent, an oligonucleotide, a radionuclide, a topoisomerase inhibitor, a tyrosine kinase inhibitor, or another agent. In some embodiments, the nanoparticles of the invention are used to transport doxorubicin to and accumulate doxorubicin in a glioblastoma tumor. In some embodiments, the nanoparticles of the invention are used to transport paclitaxel to and accumulate paclitaxel in a glioblastoma tumor. In some embodiments, the nanoparticles of the invention are used to transport auristatins to and accumulate auristatins in a glioblastoma tumor.

In some embodiments, nanoparticle drug conjugates of the invention are used to treat glioblastoma tumors. In some embodiments, the nanoparticle drug conjugates of the invention preferentially release drug at the glioblastoma tumor. In some embodiments, the nanoparticle drug conjugates are capable of delivering a therapeutically effective amount of drug to a glioblastoma tumor. In some embodiments, the nanoparticle drug conjugate delivers to a glioblastoma tumor a therapeutically effective amount of a mitotic inhibitor, an antitumor antibiotic, an immunomodulating agent, a gene therapy agent, an alkylating agent, an antiangiogenic agent, an antimetabolite, a chemoprotective agent, an antihormone agent, a corticosteroid, a photoactive therapeutic agent, an oligonucleotide, a radionuclide, a topoisomerase inhibitor, a tyrosine kinase inhibitor, or another agent. In some embodiments, the nanoparticle drug conjugates are capable of delivering a therapeutically effective amount of doxorubicin to a glioblastoma tumor. In some embodiments, the nanoparticle drug conjugates are capable of delivering a therapeutically effective amount of paclitaxel to a glioblastoma tumor. In some embodiments, the nanoparticle drug conjugates are capable of delivering a therapeutically effective amount of an auristatin to a glioblastoma tumor.

### DETAILED DESCRIPTION OF THE INVENTION

In some embodiments, the present invention can be utilized in a variety of conditions. The configuration of a nanoparticle of the invention may depend on the use for which the nanoparticle is intended. It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

It must be noted that as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation. Numerical limitations given with respect to concentrations or levels of a substance are intended to be approximate, unless the context clearly dictates otherwise. Thus, where a concentration is indicated to be (for example) 10 μg, it is intended that the concentration be understood to be at least approximately or about 10 μg.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

### Definitions

As used herein, the term “administering” includes routes of administration which allow the compositions of the invention to perform their intended function. A variety of routes of administration are possible including, but not necessarily limited to parenteral (e.g., intravenous, intra-arterial, intramuscular, subcutaneous injection), oral (e.g., dietary), topical, nasal, rectal, or via slow releasing microcarriers depending on the disease or condition to be treated. Oral, parenteral and intravenous administration are preferred modes of administration. Formulation of the compound to be administered will vary according to the route of administration selected (e.g., solution, emulsion, gels, aerosols, capsule). An appropriate composition comprising the compound to be administered can be prepared in a physiologically acceptable vehicle or carrier and optional adjuvants and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers (See generally, Remington's Pharmaceutical Science, 18th Edition, Mack, Ed. (1990), which is incorporated by reference in its entirety for all purposes).

As used herein, an “antibody” is defined to be a protein functionally defined as a ligand-binding protein and structurally defined as comprising an amino acid sequence that is recognized by one of skill as being derived from the variable region of an immunoglobulin. An antibody can consist of one or more polypeptides substantially encoded by immunoglobulin genes, fragments of immunoglobulin genes, hybrid immunoglobulin genes (made by combining the genetic information from different animals), or synthetic immunoglobulin genes. The recognized, native, immunoglobulin genes include the kappa, lambda, alpha, gamma, delta, epsilon and mu constant region genes, as well as myriad immunoglobulin variable region genes and multiple D-segments and J-segments. Light chains are classified as either kappa or lambda. Heavy chains are classified as gamma, mu, alpha, delta, or epsilon, which in turn define the immunoglobulin classes, IgG, IgM, IgA, IgD and IgE, respectively. Antibodies exist as intact immunoglobulins, as a number of well characterized fragments produced by digestion with various peptidases, or as a variety of fragments made by recombinant DNA technology. Antibodies can derive from many different species (e.g., rabbit, sheep, camel, human, or rodent, such as mouse or rat), or can be synthetic. Antibodies can be chimeric, humanized, or humaneered. Antibodies can be monoclonal or polyclonal, multiple or single chained, fragments or intact immunoglobulins.

As used herein, an “antibody fragment” is defined to be at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or VH), camelid VHH domains, and multi-specific antibodies formed from antibody fragments. The term “scFv” is defined to be a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

As used herein, an “antigen” is defined to be a molecule that provokes an immune response. This immune response may involve either antibody production, or the activation of specific immunologically-competent cells, or both. The skilled artisan will understand that any macromolecule, including, but not limited to, virtually all proteins or peptides, including glycosylated polypeptides, phosphorylated polypeptides, and other post-translation modified polypeptides including polypeptides modified with lipids, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be synthesized or can be derived from a biological sample, or can be a macromolecule besides a polypeptide. Such a biological sample can include, but is not limited to a tissue sample, a tumor sample, a cell or a fluid with other biological components.

As used herein, the term “antitumor antibiotic” means an antineoplastic drug that blocks cell growth by interfering with DNA, and is made from a microorganism. In some embodiments, antitumor antibiotics either break up DNA strands or slow down or stop DNA synthesis.

As used herein, a “cylindrical nanoparticle,” “nanoparticle cylinder,” “nanoparticle rod,” or “rod-shaped nanoparticle”, “rod-like shaped nanoparticle”, “nanorods”, “nanotubes”, “nano-wires”, “worm-like nanoparticles”, “helical symmetric nanoparticles”, “nano-worms”, “nanopencils”, “linear nano-chains”, “stretched ellipsoid shaped nanoparticles”, “nano-phage”, “micro-phage” are used interchangeably and refer to nanoparticles that have a generally cylindrical shape and can be flexible or rigid.

As used herein, “nano-discs”, “nano-cans”, “shortened or truncated nano-cylinders”, “stacked pancake-like” discs, “flattened spheres”, “flattened spheroids”, “stretched or elongated spheroids”, “ellipsoids”, nano-rings”, “ring-like” or “doughnut-like” nanoparticles are used interchangeably and refer to nanoparticles that have a generally disc or ring shape and can be flexible or rigid, symmetric or not in diameter.

As used herein, the term “cytokine” generally refers to proteins released by one cell population which act on another cell as intercellular mediators. In some embodiments, cytokines directly stimulate immune effector cells and stromal cells at the tumor site and enhance tumor cell recognition by cytotoxic effector cells. Lee and Margolin (2011) Cancers 3:3856, which is incorporated by reference in its entirety for all purposes.

As used herein, the term an “effective amount” or “therapeutically effective amount” are used interchangeably, and refer to be an amount of a compound, formulation, material, or composition, as described herein effective to achieve a particular biological result.

As used herein, the term “immunomodulating agent” refers to an agent that can stimulate or modify an immune response. In some embodiments, an immunomodulating agent is an immunostimulator which enhances a subject's immune response. In some embodiments, an immunomodulating agent is an immunosuppressant which prevents or decreases a subject's immune response. In some embodiments, an immunomodulating agent may modulate myeloid cells (monocytes, macrophages, dendritic cells, megakaryocytes and granulocytes) or lymphoid cells (T cells, B cells and natural killer (NK) cells) and any further differentiated cell thereof.

As used herein, the term “linker” refers to a chemical moiety that may be bifunctional or multifunctional, and is used to attach an agent to a nanoparticle. A linker may include one conjugating component or may include multiple components. For example, the linker may include a spacer, which is a moiety that extends the linker. Other examples of components for a linker include a stretcher unit, an amino acid unit, a self-immolative spacer, and/or a hydrophilic polymer (e.g., PEG).

As used herein, the term “mitotic inhibitor” refers to a cytotoxic and/or therapeutic agent that blocks mitosis or cell division, a biological process particularly important to glioblastoma cancer cells. A mitotic inhibitor may disrupt microtubules such that cell division is prevented, often by affecting microtubule polymerization or microtubule depolymerization. Thus, in one embodiment, a nanoparticle of the invention is conjugated to one or more mitotic inhibitor(s) that disrupts microtubule formation by inhibiting tubulin polymerization.

As used herein, the term “nanoparticle-drug-conjugate” or “NDC” refers to a nanoparticle chemically linked to one or more chemical agent(s) that may optionally be a reporter, a therapeutic agent, or a cytotoxic agent. In a preferred embodiment, a NDC includes a nanoparticle, a reporter, a cytotoxic agent, or a therapeutic drug, and a linker that enables attachment or conjugation of the agent to the nanoparticle. A NDC typically has anywhere from 1 to 8 agents conjugated to the nanoparticle, including agent loaded species of 1, 2, 3, 4, 5, 6, 7, or 8. A NDC may have a large number of reporters, cytotoxic agents, and/or therapeutic drugs conjugated to the nanoparticle. Non-limiting examples of agents that may be included in the NDCs are reporters, mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

The terms “isolated,” “purified” or “biologically pure” refer to material which is substantially or essentially free from components which normally accompany the material as it is found in its native state. Thus, isolated peptides in accordance with the invention preferably do not contain materials normally associated with the peptides in their in situ environment. An “isolated” region of a polypeptide refers to a region that does not include the whole sequence of the polypeptide from which the region was derived. An “isolated” nucleic acid, protein, or respective fragment thereof has been substantially removed from its in vivo environment so that it may be manipulated by the skilled artisan, such as but not limited to, nucleotide sequencing, restriction digestion, site-directed mutagenesis, and subcloning into expression vectors for a nucleic acid fragment as well as obtaining the protein or protein fragment in substantially pure quantities.

As used herein, the term “pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject. An example of a pharmaceutically acceptable carrier is buffered normal saline (0.15M NaCl). The use of such media and agents for pharmaceutically active substances is well known in the art. Except insofar as any conventional media or agent is incompatible with the therapeutic compound, use thereof in the compositions suitable for pharmaceutical administration is contemplated. Supplementary active compounds can also be incorporated into the compositions.

As used herein, the term “pharmacologic activity” means the effect of a drug or other chemical on a biological system. The effect of chemical may be beneficial (therapeutic) or harmful (toxic). The pure chemicals or mixtures may be of natural origin (plant, animal, or mineral) or may be synthetic compounds.

As used herein, the term “radiosensitizer” refers to a molecule, preferably a low molecular weight molecule, administered to animals in therapeutically effective amounts to increase the sensitivity of the cells to be radiosensitized to electromagnetic radiation and/or to promote the treatment of diseases that are treatable with electromagnetic radiation. Radiosensitizers are agents that make cancer cells more sensitive to radiation therapy, while typically having much less of an effect on normal cells.

As used herein, the term “reporter” or “reporter molecule” refers to a moiety capable of being detected indirectly or directly. Reporters include, without limitation, a chromophore, a fluorophore, a fluorescent protein, a receptor, a hapten, an enzyme, and a radioisotope.

As used herein, the term “reporter probe” refers to a molecule that contains a detectable label and is used to detect the presence (e.g., expression) of a reporter molecule. The detectable label on the reporter probe can be any detectable moiety, including, without limitation, an isotope, chromophore, and fluorophore. The reporter probe can be any detectable molecule or composition that binds to or is acted upon by the reporter to permit detection of the reporter molecule.

As used herein, the term “taxane” refers to the class of antineoplastic agents having a mechanism of microtubule action and having a structure that includes the taxane ring structure and a stereospecific side chain that is required for cytostatic activity. Also included within the term “taxane” are a variety of known derivatives, including both hydrophilic derivatives, and hydrophobic derivatives.

As used herein, the term “topoisomerase inhibitor” refers to chemotherapy agents designed to interfere with the action of topoisomerase enzymes (topoisomerase I and II), which are enzymes that control the changes in DNA structure by catalyzing the breaking and rejoining of the phosphodiester backbone of DNA strands during the normal cell cycle.

A used herein, the term “treatment” includes preventing, lowering, stopping, or reversing the progression or severity of the condition or symptoms associated with a condition being treated. As such, the term “treatment” includes medical, therapeutic, and/or prophylactic administration, as appropriate. Treatment may also include preventing or lessening the development of a condition, such as cancer.

### Nanoparticles

In some embodiments, the nanoparticles of the invention reach and accumulate in glioblastoma tumors. In some embodiments, the nanoparticles of the invention are designed to extravasate from tumor blood vessels and diffuse into glioblastoma tumors. In some embodiments, the average pore size for glioblastoma tumor blood vessels is about 10-50 nanometers. In some embodiments, the average pore size for glioblastoma tumor blood vessels is about 35 nanometers. In some embodiments, the average pore size for glioblastoma tumor blood vessels is about 12 nanometers. In some embodiments, the nanoparticle cylinders have a diameter of 5 to 20 nanometers, and a length of 10 to 150 nanometers. In some embodiments, the nanoparticle cylinders have a diameter of 5 to 20 nanometers, and a length of 10 to 100 nanometers. In some embodiments, the nanoparticles cylinders and have a diameter of about 5 to 10 nanometers and a length of about 25 to 50 nanometers. In some embodiments, the nanoparticle disks have a diameter of 5-40 nanometers and a thickness of about 5 to 25 nanometers. In some embodiments, the nanoparticles disks have a diameter of about 10-20 nanometers and a thickness of about 5 to 15 nanometers.

In some embodiments, the nanoparticles are viral nanoparticles. In some embodiments, viral nanoparticles (VNP) are nanoparticle formulations that can be used as building blocks for novel materials with a variety of properties. In some embodiments, viral nanoparticles are derived from bacteriophages, plant viruses, or animal viruses, and the viral nanoparticles can be infectious or non-infectious. In some embodiments, virus-like particles (VLP) are viral nanoparticles lacking any virus genomic nucleic acid, rendering them non-infectious. In some embodiments, viral nanoparticles are dynamic, self-assembling systems that form highly symmetrical, polyvalent and monodisperse structures. In some embodiments, viral nanoparticles are biocompatible and biodegradable. In some embodiments, VNPs derived from plant viruses or bacteriophages are particularly advantageous because they are less likely to be pathogenic in humans, and therefore less likely to induce undesirable side effects. Steinmetz et al., Nanomedicine. 2010 Oct; 6(5): 634-641, which is incorporated by reference in its entirety for all purposes.

In some embodiments, nanoparticle disks of the invention are made from viral nanoparticles. In some embodiments, nanoparticle disks with a diameter of 18 nanometers and a height of 5 nanometers are made using the methods described in, for example, Dedeo et al., Nanoletters 10:181-186 (2010), and Witus et al., Acc. Chem. Res. 44:774-783 (2011), which are both incorporated by reference in their entirety for all purposes. In some embodiments, viral nanoparticles are made with specific amino acids or peptide stretches for the conjugation of linkers and/or agents to the viral nanoparticle. Bernard et al., Front. Microbiol. 5:734 (2014), which is incorporated by reference in its entirety for all purposes. In some embodiments, viral nanoparticles are made using TMV polypeptides to make 1 coil or 2 coil nanoparticles with a diameter of about 15-21 nanometers and a height of about 2.5 to 7.5 nanometers. Chapman, Sean N, (March 2013) Plant Viruses with Rod-Shaped Virions. In: eLS. John Wiley & Sons Ltd, Chichester, which is incorporate by reference in its entirety for all purposes. Similar techniques can be used with other viruses (e.g., Virgoviridae or Tobomovirus) to make nanoparticle disks with similar diameters (15-21 nanometers) as the TMV nanoparticle disks.

In some embodiments, nanoparticle disks can be made with phospholipids to provide disks with diameters of about 8-15 nanometers. In some embodiments, the phospholipid disks are encircled by an amphipathic helical protein called a membrane scaffold protein (MSP). In some embodiments, nanoparticle disks self-assemble when detergent is slowly removed from a solubilized mixture of lipid and MSP Membrane. In some embodiments, the phospholipid nanoparticle disks also include membrane proteins, for example, membrane attached antibody can be included in the disks. In some embodiments, nanoparticle disks made from phospholipids may be obtained from Cube Biotech which commercially sells such nanoparticles. In some embodiments, the nanoparticle disks sold by Cube Biotech have diameters of 8-13 nanometers. Other nanoparticle disk or ring shapes are described in the Handbook of Nanophysics: Nanotubes and Nanowires (ed. K. D. Sattler), CRC Press (2010), section 3.4.4.3, which is incorporated by reference in its entirety for all purposes.

In some embodiments, nanoparticle cylinders with a diameter of 6-7 nanometers and a length of about 50 nanometers are made using, for example, the methods disclosed in Rakonjac et al. Curr. Issues Mol. Biol. 13:51-76 (2011), Specthrie et al. J. Mol. Biol. 228:720-724 (1992), Sattar et al., Front. Microbiol. 6:316 (2015), Sadia Sattar 2013, PhD Thesis Biochemistry, Massey University, NZ, titled “Filamentous phage-derived nanorods for applications in diagnostics and vaccines,” which are all incorporated by reference in their entirety for all purposes. In some embodiments, nanoparticle cylinders with a diameter of 5 nanometers and a length of about 60 nanometers are made using, for example, the methods disclosed in Tim Harrah 2008, PhD Thesis Biomedical Engineering, Tufts University, Boston Mass., titled “Engineered bacteriophage T4 tail fiber proteins for nanotechnology,” which is incorporated by reference in its entirety for all purposes. In some embodiments, these techniques are used with other bacteriophage to make a variety of cylinders of different sizes. In some embodiments, these techniques can be used with T even phages to make cylinders with diameters of about 20 nanometers and lengths from about 50 nanometers to 150 nanometers. In some embodiments, these techniques can be used with PhiX174 phage to make cylinders of about 6.5 nanometers in diameter and 25 nanometers in length. In some embodiments, these techniques are used with P22 phage to make cylinders of about 25 nanometers in diameter and 40 nanometers in length. In some embodiments, appropriate elliptical spheroids are used as nanoparticle cylinders.

In some embodiments, cylindrical or elliptical spheroid shaped nanoparticles can be made from gold nanorod particles. Pissuwan et al., Biotechnol. Genet. Engin. Rev. 25:93-112 (2008); Sigma-Aldrich sells gold nanoparticle rods with a diameter of 10 nanometers and a length of about 50 nanometers, both of these are incorporated by reference in their entirety for all purposes. In some embodiments, these gold nanoparticles can be modified by attachment of agent conjugates, and have been found to have low toxicity in biological systems. In some embodiments, nanoparticle cylinders (or nanoparticle disks) are made from polymeric materials. Tao et al., Exp. Biol. Med. 236:20-29 (2011), which is incorporated by reference in its entirety for all purposes.

In some embodiments, the nanoparticles of the invention are modified with molecules that enhance the half-life of the nanoparticle in the body of an organism. Such modifications include, for example, PEGylation, or derivatization with other hydrophilic polymers such as dextran, or other polysaccharides, PVP (polyvinylpyrrolidone), PVA (polyvinyl alcohol), etc. Derivatizing or conjugating such polymers to polypeptides (such as viral nanoparticles) are well known in the art.

In some embodiments, viral nanoparticles include antibodies that are incorporated into the nanoparticle using phage display techniques and conjugation methods as described below.

### Conjugates

In some embodiments, linkers are used to conjugate an agent to the nanoparticle. In some embodiments the linkers are multifunctional linkers. In some embodiments, the linker is stable and remains intact extracellularly and/or outside of the tumor followed by cleavage at an efficacious rate once inside the tumor and/or inside a tumor or cancer cell. In some embodiments, some linkers will: (i) maintain the specific properties of the nanoparticle important to tumor delivery; (ii) allow delivery, e.g., intracellular delivery, of the agent; and (iii) maintain the therapeutic effect, e.g., cytotoxic effect, of a drug agent. In some embodiments, the linker remains stable and intact throughout the life of the nanoparticle. In some embodiments, such stable linkers are used in diagnostic applications and the agent is an imaging reporter.

In some embodiments, linkers of the invention are based on chemical motifs including disulfides, hydrazones or peptides (cleavable), or thioethers (noncleavable) and assist in the distribution and delivery of the agents to targets. In some embodiments, the linker is cleavable or noncleavable. When the linker is noncleavable, the entire nanoparticle drug conjugate (“NDC”) may enter a target cell where the entire complex is degraded releasing the drug or agent into the cell. When a cleavable linker is used, the NDC reaches the target and there encounters conditions, polypeptides, and/or other agents that cleave the linker to release the conjugated agent (e.g., a drug).

In some embodiments, the linker is cleavable under intracellular conditions, such that cleavage of the linker sufficiently releases the agent from the nanoparticle in the intracellular or intra-tumor environment to be therapeutically effective. In some embodiments, the cleavable linker is pH-sensitive, i.e., sensitive to hydrolysis at certain pH values. In some embodiments, the pH-sensitive linker is hydrolyzable under acidic conditions found in tumors or in the lysosome. For example, an acid-labile linker that is hydrolyzable in the lysosome (e.g., a hydrazone, semicarbazone, thiosemicarbazone, cis-aconitic amide, orthoester, acetal, ketal, or the like) can be used. U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, all of which are incorporated by reference in their entirety for all purposes. Such linkers are relatively stable under neutral pH conditions, such as those in the blood, but are unstable at a pH below 5.5 or 5.0, the approximate pH of the lysosome. In some embodiments, the linker has other pH sensitive components, such as, for example, a benzoic-imine linkage (can cleave at the pH conditions found in tumors or in lysosomes), or hydrazine/hydrazone linkages. In some embodiments, the hydrolyzable linker is a thioether linker (such as, e.g., a thioether attached to the therapeutic agent via an acylhydrazone bond. U.S. Pat. No. 5,622,929, which is incorporated by reference in its entirety for all purposes). In some embodiments, the linker includes disulfide linkages that can be cleaved when the NDC is exposed to a reducing environment in a target cell or in certain vesicles in a target cell.

In some embodiments, a linker is attached to a viral nanoparticle through a thiol group (e.g., cysteine) and/or amine groups (e.g., lysine) in the viral nanoparticle polypeptide(s). In some embodiments, functional groups on the linker capable of reacting with a thiol group include, for example, maleimide, haloacetamides, α-haloacetyl, activated esters such as succinimide esters, 4-nitrophenyl esters, pentafluorophenyl esters, tetrafluorophenyl esters, anhydrides, acid chlorides, sulfonyl chlorides, isocyanates, and isothiocyanates. Klussman, et al, Bioconjugate Chemistry 15:765-773 (2004), which is incorporated by reference in its entirety for all purposes. In some embodiments, nanoparticles are synthesized to contain non-natural amino acids and agents are conjugated to the nanoparticles at these non-natural amino acids. Axup et al., Proc. Nat'l Acad. Sci. 109:16101-106 (2012), which is incorporated by reference in its entirety for all purposes.

In some embodiments, the linker has a functionality that is capable of reacting with an electrophilic group present on a nanoparticle. In some embodiments, electrophilic groups include, for example, aldehyde and ketone carbonyl groups. In some embodiments, a heteroatom of the reactive functionality of the linker can react with an electrophilic group on a nanoparticle and form a covalent bond to a nanoparticle. In some embodiment, reactive functionalities on a linker include, for example, hydrazide, oxime, amino, hydrazine, thiosemicarbazone, hydrazine carboxylate, and arylhydrazide.

In some embodiments, a linker may comprise multiple components, including one or more of 6-maleimidocaproyl, maleimidopropanoyl (“MP”), valine-citrulline (“val-cit” or “vc”), alanine-phenylalanine (“ala-phe”), p-aminobenzyloxycarbonyl (a “PAB”), N-Succinimidyl 4-(2-pyridylthio) pentanoate (“SPP”), and 4-(N-maleimidomethyl)cyclohexane-1 carboxylate (“MCC”). In some embodiments, the nanoparticle is conjugated to an auristatin, e.g., MMAE, via a linker comprising maleimidocaproyl (“mc”), valine citrulline (val-cit or “vc”), and PABA (referred to as a “mc-vc-PABA linker”). The maleimidocaproyl group acts as a linker to the nanoparticle and is not cleavable. The val-cit group is a dipeptide that is an amino acid unit of the linker and allows cleavage of the linker by a protease, specifically the protease cathepsin B. Thus, the val-cit component of the linker provides a means for releasing the auristatin from the NDC upon exposure to the intracellular environment. In some embodiments, the p-aminobenzylalcohol (PABA) group acts as a spacer (and conjugates to the MMAE) and is self immolative, allowing for the release of the MMAE. In some embodiments, the mc-vc-PABA linker may be used with many other agents.

In some embodiments, the linker may be cleavable or noncleavable. In some embodiments, cleavable linkers include acid-labile linkers (e.g., comprising hydrazone), protease-sensitive linkers, photolabile linkers, or disulfide-containing linkers. Chari et al., Cancer Research 52:127-131 (1992); U.S. Pat. No. 5,208,020, both of which are incorporated by reference in their entirety for all purposes. In some embodiments, a cleavable linker is typically susceptible to cleavage under intracellular conditions. In some embodiments, suitable cleavable linkers include, for example, a peptide linker cleavable by an intracellular protease, such as lysosomal protease or an endosomal protease. In some embodiments, the linker can include a peptide linker of 1, 2, 3, 4, 5, 6, 7, 8, 9, or 10 amino acid residues. In some embodiments, exemplary enzymatically-cleavable peptide sequences of the invention include, for example: Gly-Gly, Phe-Lys, Val-Lys, Phe-Phe-Lys, D-Phe-Phe-Lys, Gly-Phe-Lys, Ala-Lys, Val-Cit, Phe-Cit, Leu-Cit, Ile-Cit, Trp-Cit, Phe-Ala, Ala-Phe, Gly-Gly-Gly, Gly-Ala-Phe, Gly-Val-Cit, Gly-Phe-Leu-Gly, Ala-Leu-Ala-Leu, Phe-N⁹-tosyl-Arg, and Phe-N⁹-Nitro-Arg, in either orientation. In some embodiments, cleavable peptide sequences suitable for use in the present invention can be designed and optimized in their selectivity for enzymatic cleavage by a particular intracellular enzyme e.g. a tumor-associated protease. In some embodiments, cleavable peptides for use in the present invention include those which are optimized toward the proteases, cathepsin B, C and D, such as Phe-Lys, Ala-Phe, and Val-Cit. In some embodiments, the amino acid unit is the tripeptide D-Ala-Phe-Lys, which is selectively recognized by the tumor-associated protease plasmin, which is involved in tumor invasion and metastasis. de Groot, et al (2002) Molecular Cancer Therapeutics 1(11):901-911; de Groot, et al (1999) J. Med. Chem. 42(25):5277-5283, all of which are incorporated by reference in their entirety for all purposes.

In some embodiments, the linker includes a spacer unit which is a divalent moiety that couples the N-terminus of the cleavable peptide to the agent. In some embodiments the spacer unit is of a length that enables the cleavable peptide sequence to be contacted by the cleaving enzyme (e.g. cathepsin B) followed by hydrolysis of the amide bond coupling the cleavable peptide to the self-immolative spacer. In some embodiments, the agent and the self-immolative spacer form an amide bond that upon proteolytic cleavage initiates the self-immolative reaction and the ultimate release of the agent.

In some embodiments, methods used for conjugating agents to antibodies are used to conjugate agents to the nanoparticles of the invention. For example, methods can be used as those described in Nolting, Methods. Molc. Biol. 1045:71-100 (2013); Ducry et al., Bioconjug. Chem. 21:5-13 (2010), Bernard et al., Front. Microbiol. 5:734 (2014), which are all incorporated by reference in their entirety for all purposes. In some embodiments, agents and/or linkers are conjugated to nanoparticles of the invention using the conjugation chemistries and reactive groups disclosed in the Nektar Advanced PEGylation Catalog (2005-2006), which is incorporated by reference in its entirety for all purposes.

In some embodiments, multiple agents are conjugated to the nanoparticle through multiple linkers. In some embodiments, antibodies are linked to the nanoparticle to target the nanoparticle to glioblastoma cells.

In some embodiments, antibodies or other polypeptides are linked to viral nanoparticles using the recombinant methods from phage display (which are well known in the art, and some of which are described below). In some embodiments, viral nanoparticles are made with a fusion protein at the carboxyl terminus of a coat protein that provides a cysteine or other amino acid for chemical ligation to another polypeptide or linker. Dwyer et al., Chem. Biol. 7:263-274 (2000). In some embodiments, viral nanoparticles are engineered to include an acceptor peptide substrate for yeast biotin ligase (the yeast enzyme attaches a biotin at the acceptor peptide sequence) to produce viral nanoparticles labeled with biotin. Chen et al., J. Am. Chem. Soc. 129:6619-6625 (2007), which is incorporated by reference in its entirety for all purposes. In this embodiment, agents of choice or antibodies or other molecules may be conjugated to the viral nanoparticle using the biotin moiety and, for example, the interaction of streptavidin with biotin. In some embodiments, well known chemistries for labeling the δ (delta) amino group of lysine are used to attach linkers, biotin, polypeptides, or agents to viral nanoparticles. Jin et al., Biotechniques 46:175-182 (2009). In some embodiments, the amino terminal end of the coat proteins from a viral nanoparticle are converted to ketone groups for use in conjugation of agents or antibodies to the viral nanoparticle. Carrico et al., ACS Nano 6:6675-6680 (2012), which is incorporated by reference in its entirety for all purposes. In some embodiments, viral nanoparticles are modified using the methods and techniques described in Bernard et al., Front. Microbiol. 5:734 (2014), which is incorporated by reference in its entirety for all purposes.

### Agents

Agents for conjugation to the nanoparticles of the invention can be any agents useful for diagnostics or therapy of glioblastoma. In some embodiments, the agent is a reporter as disclosed below. In some embodiments, the agent is a mitotic inhibitor, antitumor antibiotics, immunomodulating agents, gene therapy agents, alkylating agents, antiangiogenic agents, antimetabolites, chemoprotective agents, antihormone agents, corticosteroids, photoactive therapeutic agents, oligonucleotides, radionucleotides, topoisomerase inhibitors, tyrosine kinase inhibitors, or other agents.

In one aspect, nanoparticles of the invention may be conjugated to one or more mitotic inhibitor(s) to form an NDC for the treatment of glioblastoma. In some embodiments, the mitotic inhibitor used in the NDCs of the invention include, for example, Ixempra (ixabepilone), vincristine, auristatins, dolastatins, maytansinoids, or plant alkaloids.

Auristatins represent a group of dolastatin analogs that have generally been shown to possess anticancer activity by interfering with microtubule dynamics and GTP hydrolysis, thereby inhibiting cellular division. For example, Auristatin E (U.S. Pat. No. 5,635,483, which is incorporated by reference in its entirety for all purposes) is a synthetic analogue of the marine natural product dolastatin 10, a compound that inhibits tubulin polymerization by binding to the same site on tubulin as the anticancer drug vincristine (G. R. Pettit, Prog. Chem. Org. Nat. Prod, 70: 1-79 (1997), which is incorporated by reference in its entirety for all purposes). Dolastatin 10, auristatin PE, and auristatin E are linear peptides having four amino acids, three of which are unique to the dolastatin class of compounds. In some embodiments, the auristatin subclass of mitotic inhibitors include, but are not limited to, monomethyl auristatin D (MMAD or auristatin D derivative), monomethyl auristatin E (MMAE or auristatin E derivative), monomethyl auristatin F (MMAF or auristatin F derivative), auristatin F phenylenediamine (AFP), auristatin EB (AEB), auristatin EFP (AEFP), and 5-benzoylvaleric acid-AE ester (AEVB). The synthesis and structure of auristatin derivatives are described in U.S. Patent Application Publication Nos. 2003-0083263, 2005-0238649 and 2005-0009751; International Patent Publication No. WO 04/010957, International Patent Publication No. WO 02/088172, and U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference in its entirety for all purposes.

In some embodiments, nanoparticles of the invention are conjugated to at least one MMAE (mono-methyl auristatin E). Monomethyl auristatin E (MMAE, vedotin) inhibits cell division by blocking the polymerization of tubulin. In some embodiments, the linker linking MMAE to the nanoparticle is stable in extracellular fluid (i.e., the medium or environment that is external to cells), but is cleaved by cathepsin once the NDC has entered a cancer cell, thus releasing the toxic MMAE and activating the potent anti-mitotic mechanism.

In some embodiments, Dolastatins are used as antimitotic agents for conjugation to nanoparticles of the invention. In some embodiments, Dolastatins are short peptidic compounds isolated from the Indian Ocean sea hare Dolabella auricularia. Pettit et al., J. Am. Chem. Soc., 1976, 98, 4677, which is incorporated by reference in its entirety for all purposes. In some embodiments, dolastatins include dolastatin 10 and dolatstin 15. Dolastatin 15, a seven-subunit depsipeptide derived from Dolabella auricularia, is a potent antimitotic agent structurally related to the antitubulin agent dolastatin 10, a five-subunit peptide obtained from the same organism. In some embodiments, auristatins, described above, are synthetic derivatives of dolastatin 10.

In some embodiments, nanoparticles of the invention are conjugated to maytansinoids. In some embodiments, maytansinoids are isolated from members of the higher plant families Celastraceae, Rhamnaceae and Euphorbiaceae, as well as some species of mosses. Kupchan et al, J. Am. Chem. Soc. 94:1354-1356 (1972); Wani et al, J. Chem. Soc. Chem. Commun. 390: (1973); Powell et al, J. Nat. Prod. 46:660-666 (1983); Sakai et al, J. Nat. Prod. 51:845-850 (1988); Suwanborirux et al, Experientia 46:117-120 (1990), U.S. Pat. No. 6,441,163, which are all incorporated by reference in their entirety for all purposes. In some embodiments, maytansinoids include maytansine, maytansinol, C-3 esters of maytansinol, and other maytansinol analogues and derivatives. U.S. Pat. Nos. 5,208,020 and 6,441,163, which are incorporated by reference in their entirety for all purposes. In some embodiments, naturally occurring and synthetic C-3 maytansinol esters can be classified as C-3 esters with simple carboxylic acids, or C-3 esters with derivatives of N-methyl-L-alanine, the latter being more cytotoxic than the former. Synthetic maytansinoid analogues are described in, for example, Kupchan et al., J. Med. Chem., 21, 31-37 (1978), which is incorporated by reference in its entirety for all purposes. In some embodiments, maytansinoids include, for example, DM1 (N²′-deacetyl-N²′-(3-mercapto-1-oxopropyl)-maytansine; also referred to as mertansine, drug maytansinoid 1, ImmunoGen, Inc (Chari et al. Cancer Res 52:127 (1992), which is incorporated by reference in its entirety for all purposes), DM2, DM3 (N²′-deacetyl-N²′-(4-mercapto-1-oxopentyl)-maytansine), DM4 (4-methyl-4-mercapto-1-oxopentyl)-maytansine), maytansinol (a synthetic maytansinoid analog), ansamitocin P1, ansamitocin P2, ansamitocin P3, and ansamitocin P4. Other examples of maytansinoids are described in U.S. Pat. No. 8,142,784, which is incorporated by reference in its entirety for all purposes.

In some embodiments, nanoparticles of the invention are conjugated to plant alkaloids. In some embodiments, the plant alkaloid is a vinca alkaloid made from the periwinkle plant (catharanthus rosea), or a taxane made from the bark of the Pacific Yew tree (taxus). In some embodiments, vinca alkaloids of the invention include, but are not limited to vindesine sulfate, vincristine, vinblastine and vinorelbine.

In some embodiments, taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869, all of which are incorporated by reference in their entirety for all purposes. In some embodiments, taxane compounds include those previously described in U.S. Pat. Nos. 5,641,803, 5,665,671, 5,380,751, 5,728,687, 5,415,869, 5,407,683, 5,399,363, 5,424,073, 5,157,049, 5,773,464, 5,821,263, 5,840,929, 4,814,470, 5,438,072, 5,403,858, 4,960,790, 5,433,364, 4,942,184, 5,362,831, 5,705,503, and 5,278,324, all of which are incorporated by reference in their entirety for all purposes. Further examples of taxanes include, but are not limited to, docetaxel (Taxotere; Sanofi Aventis), and paclitaxel (Abraxane or Taxol; Abraxis Oncology).

In some embodiments, nanoparticles of the invention are conjugated to antitumor antibiotics. In some embodiments, antitumor antibiotics include, but are not limited to, actinomycines (e.g., pyrrolo[2,1-c][1,4]benzodiazepines), anthracyclines, calicheamicins, and duocarmycins.

Actinomycines are a subclass of antitumor antibiotics isolated from bacteria of the genus Streptomyces. In some embodiments, actinomycines include, for example, actinomycin D (also known as actinomycin, dactinomycin, actinomycin IV, actinomycin Cl, Lundbeck, Inc.), anthramycin, chicamycin A, DC-81, mazethramycin, neothramycin A, neothramycin B, porothramycin, prothracarcin B, SG2285, sibanomicin, sibiromycin and tomaymycin. In some embodiments, actinomycine is a pyrrolobenzodiazepine (PBD). In some embodiments, PBDs include, for example, anthramycin, chicamycin A, DC-81, mazethramycin, neothramycin A, neothramycin B, porothramycin, prothracarcin B, SG2000 (SJG-136), SG2202 (ZC-207), SG2285 (ZC-423), sibanomicin, sibiromycin and tomaymycin. U.S. Pat. No. 7,741,319, U.S. Patent Application Pub. Nos. 2015/0337042, 2013/0028917 and 2013/0028919, WO 2011/130598, and WO 2006/111759, all of which are incorporated by reference in their entirety for all purposes.

In some embodiments, anthracyclines isolated from bacteria of the genus Streptomyces are used as antitumor antibiotics. In some embodiments, anthracyclines include, for example, daunorubicin (Cerubidine, Bedford Laboratories), doxorubicin (Adriamycin, Bedford Laboratories; also referred to as doxorubicin hydrochloride, hydroxydaunorubicin, and Rubex), epirubicin (Ellence, Pfizer), and idarubicin (Idamycin; Pfizer Inc.).

In some embodiments, the antitumor antibiotic is a calicheamicins derived from the soil organism Micromonospora echinospora. In some embodiments, calicheamicins that may be used in drug conjugates with nanoparticles of the invention are described, for example, in U.S. Pat. Nos. 5,712,374; 5,714,586; 5,739,116; 5,767,285; 5,770,701; 5,770,710; 5,773,001; and 5,877,296, which are incorporated by reference in their entirety for all purposes. In some embodiments, structural analogues of calicheamicin include, for example, γ₁¹, α₂¹, α₃¹, N-acetylγ₁², PSAG and θ¹₁. Hinman et al., Cancer Research 53:3336-3342 (1993), Lode et al., Cancer Research 58:2925-2928 (1998) and U.S. Pat. Nos. 5,712,374; 5,714,586; 5,739,116; 5,767,285; 5,770,701; 5,770,710; 5,773,001; and 5,877,296, which are all incorporated by reference in their entirety for all purposes.

In some embodiments, the antitumor antibiotic is a duocarmycins. In some embodiments, the duocarmycins include, for example, adozelesin, bizelesin, and carzelesin. Other antitumor antibiotics that can be used in the invention include for example, bleomycin (Blenoxane, Bristol-Myers Squibb), mitomycin, and plicamycin (also known as mithramycin).

In some embodiments, nanoparticles of the invention are conjugated to immunomodulating agents. In some embodiments, immunomodulating agents include, for example, bacillus calmette-guerin (BCG), levamisole (Ergamisol), cancer vaccines, cytokines, and immunomodulating gene therapy.

In some embodiments, the immunomodulating agent is a cytokine. In some embodiments, cytokines for use in conjugates with nanoparticles include, for example, parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor; mullerian-inhibiting substance; mouse gonadotropin-associated peptide; inhibin; activin; vascular endothelial growth factor; integrin; thrombopoietin (TPO); nerve growth factors such as NGF; platelet-growth factor; transforming growth factors (TGFs); insulin-like growth factor-I and -II; erythropoietin (EPO); osteoinductive factors; interferons such as interferon α, β, and γ, colony stimulating factors (CSFs); granulocyte-macrophage-C-SF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; tumor necrosis factor; and other polypeptide factors including LIF and kit ligand (KL).

In some embodiments, nanoparticles of the invention are conjugated to alkylating agents. In some embodiments, alkylating agents that include, for example, alkyl sulfonates, ethylenimimes, methylamine derivatives, epoxides, nitrogen mustards, nitrosoureas, triazines, and hydrazines. In some embodiments, alkylating agents include, for example, busulfan (Myleran, GlaxoSmithKline; Busulfex IV, PDL BioPharma, Inc.), chlorambucil (Leukeran, GlaxoSmithKline), cyclophosphamide (Cytoxan, Bristol-Myers Squibb; Neosar, Pfizer, Inc.), estramustine (estramustine phosphate sodium or Estracyt), Pfizer, Inc.), ifosfamide (Ifex, Bristol-Myers Squibb), mechlorethamine (Mustargen, Lundbeck Inc.), melphalan (Alkeran or L-Pam or phenylalanine mustard; GlaxoSmithKline), carmustine (BCNU [also known as BiCNU, N,N-Bis(2-chloroethyl)-N-nitrosourea, or 1,3-bis(2-chloroethyl)-1-nitrosourea], Bristol-Myers Squibb), fotemustine (also known as Muphoran), lomustine (CCNU or 1-(2-chloro-ethyl)-3-cyclohexyl-1-nitrosourea, Bristol-Myers Squibb), nimustine (also known as ACNU), streptozocin (Zanosar, Teva Pharmaceuticals), dacarbazine (DTIC-Dome, Bayer Healthcare Pharmaceuticals Inc.), procarbazine (Mutalane, Sigma-Tau Pharmaceuticals, Inc.), temozolomide (Temodar, Schering Plough), thiopeta (Thioplex, Amgen), diaziquone (also known as aziridinyl benzoquinone (AZQ)), and mitomycin C.

In some embodiments, nanoparticles of the invention are conjugated to antiangiogenic agents. Antiangiogenic agents inhibit the growth of new blood vessels. In some embodiments, antiangiogenic agents interfere with the ability of a growth factor to reach its target. For example, vascular endothelial growth factor (VEGF) is one of the primary proteins involved in initiating angiogenesis by binding to particular receptors on a cell surface. In some embodiments, certain antiangiogenic agents prevent the interaction of VEGF with its cognate receptor inhibiting the initiation of angiogenesis. In some embodiments, the antiangiogenic agents interfere with intracellular signaling cascades. For example, once a particular receptor on a cell surface has been triggered, a cascade of other chemical signals is initiated to promote the growth of blood vessels. Thus, certain enzymes, for example, some tyrosine kinases are targets for cancer treatment. In some embodiments, these agents disable specific targets that activate and promote cell growth or directly interfere with the growth of blood vessel cells. In some embodiments, antiangiogenic agents include, for example, cilengitide, angiostatin, ABX EGF, C1-1033, PKI-166, EGF vaccine, EKB-569, GW2016, ICR-62, EMD 55900, CP358, PD153035, AG1478, IMC-C225 (Erbitux, ZD1839 (Iressa), OSI-774, Erlotinib (tarceva), angiostatin, arrestin, endostatin, BAY 12-9566 and w/fluorouracil or doxorubicin, canstatin, carboxyamidotriozole and with paclitaxel, EMD121974, S-24, vitaxin, dimethylxanthenone acetic acid, IM862, Interleukin-12, Interleukin-2, NM-3, HuMV833, PTK787, RhuMab, angiozyme (ribozyme), IMC-1C11, Neovastat, marimstat, prinomastat, BMS-275291, COL-3, MM1270, SU101, SU6668, SU11248, SU5416, with paclitaxel, with gemcitabine and cisplatin, and with irinotecan and cisplatin and with radiation, tecogalan, temozolomide and PEG interferon α2b, tetrathiomolybdate, TNP-470, thalidomide, CC-5013 and with taxotere, tumstatin, 2-methoxyestradiol, VEGF trap, mTOR inhibitors (deforolimus, everolimus (Afinitor, Novartis Pharmaceutical Corporation), and temsirolimus (Torisel, Pfizer, Inc.)), tyrosine kinase inhibitors (e.g., erlotinib (Tarceva, Genentech, Inc.), imatinib (Gleevec, Novartis Pharmaceutical Corporation), gefitinib (Iressa, AstraZeneca Pharmaceuticals), dasatinib (Sprycel, Brystol-Myers Squibb), sunitinib (Sutent, Pfizer, Inc.), nilotinib (Tasigna, Novartis Pharmaceutical Corporation), lapatinib (Tykerb, GlaxoSmithKline Pharmaceuticals), sorafenib (Nexavar, Bayer and Onyx), phosphoinositide 3-kinases (PI3K).

In some embodiments, nanoparticles of the invention are conjugated to antimetabolites. In some embodiments, antimetabolites include, for example, a folic acid antagonist (e.g., methotrexate), a pyrimidine antagonist (e.g., 5-Fluorouracil, Foxuridine, Cytarabine, Capecitabine, and Gemcitabine), a purine antagonist (e.g., 6-Mercaptopurine and 6-Thioguanine) and an adenosine deaminase inhibitor (e.g., Cladribine, Fludarabine, Nelarabine and Pentostatin). In some embodiments, antifolates are a subclass of antimetabolites that are structurally similar to folate. Representative examples include, for example, methotrexate, 4-amino-folic acid (also known as aminopterin and 4-aminopteroic acid), lometrexol (LMTX), pemetrexed (Alimpta, Eli Lilly and Company), and trimetrexate (Neutrexin, Ben Venue Laboratories, Inc.). In some embodiments, purine antagonists include, for example, azathioprine (Azasan, Salix; Imuran, GlaxoSmithKline), cladribine (Leustatin [also known as 2-CdA], Janssen Biotech, Inc.), mercaptopurine (Purinethol (also known as 6-mercaptoethanol), GlaxoSmithKline), fludarabine (Fludara, Genzyme Corporation), pentostatin (Nipent, also known as 2′-deoxycoformycin (DCF)), 6-thioguanine (Lanvis (also known as thioguanine), GlaxoSmithKline). In some embodiments, pyrimidine antagonists include, for example, to azacitidine (Vidaza, Celgene Corporation), capecitabine (Xeloda, Roche Laboratories), Cytarabine (also known as cytosine arabinoside and arabinosylcytosine, Bedford Laboratories), decitabine (Dacogen, Eisai Pharmaceuticals), 5-fluorouracil (Adrucil, Teva Pharmaceuticals; Efudex, Valeant Pharmaceuticals, Inc), 5-fluoro-2′-deoxyuridine 5′-phosphate (FdUMP), 5-fluorouridine triphosphate, and gemcitabine (Gemzar, Eli Lilly and Company).

In some embodiments, nanoparticles of the invention are conjugated to radionuclides. In some embodiments, radionuclides include, for example, ¹¹¹In, ¹⁷⁷Lu, ²¹²Bi, ²¹³Bi, ²¹¹At, ⁶²Cu, ⁶⁴Cu, ⁶⁷Cu, ⁹⁰Y, ¹²⁵I, ¹³¹I, ³²P, ³³P, ⁴⁷Sc, ¹¹¹Ag, ⁶⁷Ga, ¹⁴²Pr, ¹³⁵Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸AU, and ²¹¹Pb. In some embodiments, radionuclides that substantially decay with Auger-emitting particles are used, including, for example, Co-58, Ga-67, Br-80m, Tc-99m, Rh-103m, Pt-109, In-111 1, Sb-119, 1-125, Ho-161, Os-189m and Ir-192. In some embodiments, useful beta-particle-emitting nuclides include, for example, Dy-152, At-211, Bi-212, Ra-223, Rn-219, Po-215, Bi-21 1, Ac-225, Fr-221, At-217, Bi-213 and Fm-255. In some embodiments, useful alpha-particle-emitting radionuclides have energies in the range of 2,000-10,000 keV, or 3,000-8,000 keV, or 4,000-7,000 keV. In some embodiments, potential radioisotopes of use include, for example, ¹¹C, ¹³N, ¹⁵O, ⁷⁵Br, ¹⁹⁸Au, ²²⁴Ac, ¹²⁶I, ¹³³I, ⁷⁷Br, ¹¹³In, ⁹⁵Ru, ⁹⁷Ru, ¹⁰³Ru, ¹⁰⁵Ru, ¹⁰⁷Hg, ²⁰³Hg, ^{121m}Te, ^{125m}Te, ¹⁶⁵Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵⁸Co, ⁵¹Cr, ⁵⁹Fe, ⁷⁵Se, ²⁰¹Tl, ²²⁵Ac, ⁷⁶Br, ¹⁶⁹Yb, and the like.

In some embodiments, nanoparticles of the invention are conjugated to radiosensitizers. In some embodiments, radiosensitizers include, for example, gemcitabine, 5-fluorouracil, taxane, cisplatin, metronidazole, misonidazole, desmethylmisonidazole, pimonidazole, etanidazole, nimorazole, mitomycin C, RSU 1069, SR 4233, E09, RB 6145, nicotinamide, 5-bromodeoxyuridine (BUdR), 5 ododeoxyuri dine (IUdR), bromodeoxycytidine, fluorodeoxyuridine (FUdR), hydroxyurea, and therapeutically effective analogs and derivatives of the same. In some embodiments, radiosensitizers may be activated using photodynamic therapy (PDT). In some embodiments, photodynamic radiosensitizers include, for example, hematoporphyrin derivatives, Photofrin(r), benzoporphyrin derivatives, NPe6, tin etioporphyrin (SnET2), pheoborbide a, bacteriochlorophyll a, naphthalocyanines, phthalocyanines, zinc phthalocyanine, and therapeutically effective analogs and derivatives of the same. In some embodiments, radiosensitizers include those described in D. M. Goldberg (ed.), Cancer Therapy with Radiolabeled Antibodies, CRC Press (1995), which is incorporated by reference in its entirety for all purposes.

In some embodiments, nanoparticles of the invention are conjugated to topoisomerase inhibitors. In some embodiments, DNA topoisomerase I inhibitors include, for example, edotecarin, camptothecins and its derivatives irinotecan (CPT-11, Camptosar, Pfizer, Inc.) and topotecan (Hycamtin, GlaxoSmithKline Pharmaceuticals). In some embodiments, DNA topoisomerase II inhibitors include, for example, amsacrine, daunorubicin, doxotrubicin, epipodophyllotoxins, ellipticines, epirubicin, etoposide, razoxane, and teniposide.

In some embodiments, nanoparticles of the invention are conjugated to tyrosine kinase inhibitors. In some embodiments, tyrosine kinase inhibitors include, for example, Axitinib, Bosutinib, Cediranib, Dasatinib, Erlotinib, Gefitinib, Imatinib, Lapatinib, Lestaurtinib, Nilotinib, Semaxanib, Sunitinib, and Vandetanib.

### Reporters

Any reporter that can be detected in a diagnostic application may be attached to the nanoparticles of the invention. Exemplary reporters include, for example, a fluorescent reporter, a bioluminescent reporter, an enzyme, a magnetic reporter, a Positron Emission Tomography (PET) reporter, a Single Photon Emission Computed Tomography (SPECT) reporter, an X-Ray reporter, a photoacoustic reporter, and an ultrasound reporter.

In some embodiments, the PET reporter comprises a thymidine kinase. In some embodiments, the thymidine kinase is selected from a Herpes Simplex Virus thymidine kinase, Varicella-Zoster Virus thymidine kinase, human mitochondrial thymidine kinase or active variants thereof (see, e.g., Campbell et al., J Biol Chem. 287(1):446-54 (2012)). PET detection of thymidine kinase is generally achieved by using a PET-specific reporter probe. Exemplary PET reporter probe for HSV thymidine kinase includes [18F]9-(4-[18F]-fluoro-3-hydroxymethylbutyl)-guanine, a fluorine-18-labelled penciclovir analogue, which when phosphorylated by thymidine kinase (TK) becomes retained intracellularly. Another thymidine kinase reporter probe is 5-(76) Br-bromo-2′-fluoro-2′-deoxyuridine. In some embodiments, a thymidine kinase reporter probe that is preferentially acted on by the heterologous thymidine kinase as compared to any endogenous thymidine kinase is used.

Other PET reporters include, among others, dopamine D2 (D2R) receptor, sodium iodide transporter (NIS), dexoycytidine kinase, somatostatin receptor subtype 2, norepinephrine transporter (NET), cannaboid receptor, glucose transporter (Glut1), tyrosinase, and active variants thereof. The relevant reporter probes for each of the PET reporters are well known to the skilled artisan. An exemplary reporter probe for dopamine D2 (D2R) receptor is 3-(2′-[18F]fluoroethyl)spiperone (FESP) (MacLaren et al., Gene Ther. 6(5):785-91 (1999)). An exemplary reporter probe for the sodium iodide transporter is 124I, which is retained in cells following transport by the transporter. An exemplary reporter probe for deoxycytidine kinase is 2′-deoxy-2′-18F -5-ethyl-1-β-d-arabinofuranosyluracil (18F-FEAU). An exemplary reporter probe for somatostatin receptor subtype 2 is 11In-, 99m/94mTc-, 90Y-, or 177Lu-labeled octreotide analogues, for example 90Y-, or 177Lu-labeled DOTATOC (Zhang et al., J Nucl Med. 50(suppl 2):323 (2009)); 68Ga-DOTATATE; and 111In-DOTABASS (see. e.g., Brader et al., J Nucl Med. 54(2):167-172 (2013), incorporated herein by reference). An exemplary reporter probe for norepinephrine transporter is 11C-m-hydroxyephedrine (Buursma et al., J Nucl Med. 46:2068-2075 (2005)). An exemplary reporter probe for the cannaboid receptor is 11C-labeled CB2 ligand, 11C-GW405833 (Vandeputte et al., J Nucl Med. 52(7):1102-1109 (2011)). An exemplary reporter probe for the glucose transporter is [18F]fluoro-2-deoxy-d-glucose (Herschman, H. R., Crit Rev Oncology/Hematology 51:191-204 (2004)). An exemplary reporter probe for tyrosinase is N-(2-(diethylamino)ethyl)-18F-5-fluoropicolinamide (Qin et al., Sci Rep. 3:1490 (2013)). Other reporter probes are described in the art, for example, in Yaghoubi et al., Theranostics 2(4):374-391 (2012), incorporated herein by reference.

Exemplary photoacoustic reporters include, among others, tyrosinase and β-galactosidase (see, e.g., Krumholz et al., J Biomed Optics. 16(8):1-3 (2011)). Reporter probes for tyrosinase have been described above. An exemplary reporter probe for ↑-galactosidase is 5-bromo-4-chloro-3-indolyl-β-D-galactoside (X-gal) (Li et al., J Biomed Opt. 12(2):020504 (2007)).

Exemplary X-ray reporter includes, among others, somatostatin receptor 2, or other types of receptor based binding agents. The reporter probe can have a radiopaque label moiety that is bound to the reporter probe and imaged, for example, by X-ray or computer tomography. Exemplary radiopaque label is iodine, particularly a polyiodinated chemical group (see, e.g., U.S. Pat. No. 5,141,739), and paramagnetic labels (e.g., gadolinium), which can be attached to the reporter probe by conventional means.

Exemplary ultrasound reporter includes, among others, a binding agent that is capable of binding an ultrasound contrast agent, for example, a microbubble contrast agent. For example, the binding agent can comprise an antibody directed specifically against a peptide, where the peptide is bound to a microbubble contrast agent (see, e.g., Kiessling et al., J Nucl. Med. 53:345-348 (2012)).

In some embodiments herein where the reporter is a bioluminescent reporter, any number of bioluminescent proteins can be used as the reporter. These include, by way of example and not limitation, aequorin (and other Ca⁺² regulated photoproteins), luciferase based on luciferin substrate, luciferase based on Coelenterazine substrate (e.g., Renilla, Gaussia, and Metridina), and luciferase from Cypridina, and active variants thereof.

In the embodiments herein where the reporter is a fluorescent reporter, any number of fluorescent reporters can be used. These include, for example, green fluorescent protein from Aequorea victoria or Renilla reniformis, and active variants thereof (e.g., blue fluorescent protein, yellow fluorescent protein, cyan fluorescent protein, etc.); fluorescent proteins from Hydroid jellyfishes, Copepod, Ctenophora, Anthrozoas, and Entacmaea quadricolor, and active variants thereof; and phycobiliproteins and active variants thereof.

### Antibodies for Glioblastoma

In some embodiments, antibodies specific for cell surface markers on glioblastoma tumors are used with the nanoparticles of the invention. In some embodiments, antibodies with binding specificity for CD15, SSEA1, CD133, A2B5, integrin α6, and/or podoplanin are used with the nanoparticles of the invention.

Antibodies of the invention will have binding specificity for one or more surface markers on glioblastoma tumor and/or cancer cells. In some embodiments, the antibodies have specificity for one or more of CD15, SSEA1, CD133, A2B5, integrin α6, and/or podoplanin. The antibodies of the invention encompass all forms as discussed above. In an embodiment, the antibody will be engineered for a particular organism. The organism can be a human, canine, feline, or a commercially valuable livestock, such as, for example, pigs, horses, chickens, or other birds. Such engineering of the antibody includes, for example, humanization, humaneering, chimerization, or isolating human (or other organism) antibodies using any of the repertoire technologies or monoclonal technologies known in the art.

In some embodiments, antibodies of the invention are single chain antibodies, antibody fragments, or recombinantly derived antibody fragments. Methodologies for making single chain antibodies are well known in the art and include, for example, the techniques commercially offered by Creative Biolabs, GenScript, and Abcam, as well as those described in Blazek et al., Folia Microbiol. 48:687-698 (2003), Kip;riyanov et al., J. Immunol. Methods 200:69-77 (1997), which are all incorporated by reference in their entirety for all purposes. In some embodiments, recombinant DNA technology is used to make antibody fragments for use as antibodies of the invention. Nelson, MAbs 2:77-83 (2010), which is incorporated by reference in its entirety for all purposes. In some embodiments, Fab or F(ab)₂ fragments made by enzymatic digestion of full length antibodies are used in the invention. In some embodiments, the antibodies of the invention are engineered into viral nanoparticles using techniques from phage display libraries for antibodies to place the desired antibody on the surface of the viral nanoparticle. Deantonio et al., Methods Molc. Biol. 1060:277-295 (2014), Winter et al., Ann. Rev. Immunol. 12:433-455 (1994), both of which are incorporated by reference in their entirety for all purposes. In some embodiments, the techniques and reagents commercially available are used to make phage display constructs using the antibodies of the invention. Examples of such commercially available reagents and techniques are those sold by Antibody Design Laboratories.

Established methods for the isolation of antigen-specific human antibodies include the screening of hybridomas from mice that are transgenic for the human immunoglobulin loci (e.g., Jakobavits, 1998, Adv Drug Deliv Rev. 31:33-42, which is hereby incorporated by reference in its entirety), and in vitro methods in which recombinant libraries of human antibody fragments displayed on and encoded in filamentous bacteriophage (e.g., McCafferty et al., 1990, Nature 348:552-554, which is hereby incorporated by reference in its entirety), yeast cells (e.g., Boder and Wittrup, 1997, Nat Biotechnol 15:553-557, which is hereby incorporated by reference in its entirety), and ribosomes (e.g., Hanes and Pluckthun, 1997, Proc Natl Acad Sci USA 94:4937-4942, which is hereby incorporated by reference in its entirety) are panned against immobilized antigen. These methods have yielded many useful human antibodies.

The most widely used methods for minimizing the immunogenicity of non-human antibodies while retaining specificity and affinity involve grafting the CDRs of the non-human antibody onto human frameworks typically selected for their structural homology to the non-human framework (Jones et al., 1986, Nature 321:522-5; U.S. Pat. No. 5,225,539, both of which are hereby incorporated by reference in their entirety). Originally these methods resulted in drastic losses of affinity. However, it was then shown that some of the affinity could be recovered by restoring the non-human residues at key positions in the framework that are required to maintain the canonical structures of the non-human CDRs 1 and 2 (Bajorath et al., 1995, J Biol Chem 270:22081-4; Martin et al., 1991, Methods Enzymol. 203:121-53; Al- Lazikani, 1997, J Mol Biol 273:927-48, all of which are hereby incorporated by reference in their entirety). Recovering the native conformations of CDR3s is a much more uncertain enterprise because their structures are more variable. Determining which non-human residues to restore to recover functional CDR3 conformation is thus largely a matter of modeling where possible combined with trial and error. Exemplary methods for humanization of antibodies by CDR grafting are disclosed, for example, in U.S. Pat. No. 6,180,370, which is hereby incorporated by reference in its entirety.

Improvements to the traditional CDR-grafting approaches use various hybrid selection approaches, in which portions of the non-human antibody have been combined with libraries of complementary human antibody sequences in successive rounds of selection for antigen binding, in the course of which most of the non-human sequences are gradually replaced with human sequences. For example, in the chain-shuffling technique (Marks, et al., 1992, Biotechnology 10:779-83, which is hereby incorporated by reference in its entirety) one chain of the non-human antibody is combined with a naive human repertoire of the other chain on the rationale that the affinity of the non-human chain will be sufficient to constrain the selection of a human partner to the same epitope on the antigen. Selected human partners are then used to guide selection of human counterparts for the remaining non-human chains.

Other methodologies include chain replacement techniques where the non-human CDR3s were retained and only the remainder of the V-regions, including the frameworks and CDRs 1 and 2, were individually replaced in steps performed sequentially (e.g., U.S. Patent Application No. 20030166871; Rader, et al., Proc Natl Acad Sci USA 95:8910-15, 1998; Steinberger, et al., J. Biol. Chem. 275:36073-36078, 2000; Rader, et al., J. Biol. Chem. 275:13668-13676, 2000, all of which are hereby incorporated by reference in their entirety).

### Pharmaceutical Compositions

Pharmaceutical compositions of the present invention may comprise a nanoparticle or nanoparticle conjugate of the invention, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

Suitable pharmaceutically acceptable excipients are well known to a person skilled in the art. Examples of the pharmaceutically acceptable excipients include phosphate buffered saline (e.g. 0.01 M phosphate, 0.138 M NaCl, 0.0027 M KCl, pH 7.4), an aqueous solution containing a mineral acid salt such as a hydrochloride, a hydrobromide, a phosphate, or a sulfate, saline, a solution of glycol or ethanol, and a salt of an organic acid such as an acetate, a propionate, a malonate or a benzoate. In some embodiments, an adjuvant such as a wetting agent or an emulsifier, and a pH buffering agent can also be used. In some embodiments, the pharmaceutically acceptable excipients described in Remington's Pharmaceutical Sciences, 18^{th} Edition (Mack Pub. Co., N.J. 1990) and Remington: The Science and Practice of Pharmacy, 22^{nd} Edition (Mack Pub. Co., N.J. 2012) (which is incorporated herein by reference in its entirety for all purposes) can be appropriately used. The composition of the present invention can be formulated into a known form suitable for parenteral administration, for example, injection or infusion. In some embodiments, the composition of the present invention may comprise formulation additives such as a suspending agent, a preservative, a stabilizer and/or a dispersant, and a preservation agent for extending a validity term during storage.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, intranasally, intraarterially, intratumorally, into an afferent lymph vessel, by intravenous (i.v.) injection, or intraperitoneally. In one aspect, the nanoparticle compositions of the present invention are administered to a patient by intradermal or subcutaneous injection. In one aspect, the nanoparticle compositions of the present invention are administered by i.v. injection. The compositions of nanoparticle compositions may be injected directly into a tumor, lymph node, or site of infection.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). In some embodiments, a pharmaceutical composition comprising the nanoparticles described herein may be administered at a dosage of 0.001 mg/kg/day to about 500 mg/kg/day, in some instances 0.1 mg/kg/day to about 100 mg/kg/day, including all values within those ranges. In some embodiments, a nanoparticle composition may also be administered multiple times at these dosages. In some embodiments, nanoparticles can be administered by using infusion techniques that are commonly known in immunotherapy (see, e.g., Rosenberg et al., New Eng. J. of Med. 319:1676, 1988, which is incorporated by reference in its entirety for all purposes).

### Methods of Using the Nanoparticles

In some embodiments, the nanoparticles of the invention are used in methods of delivering agents to a glioblastoma tumor. In some embodiments, the nanoparticles of the invention are administered systemically to a subject. In some embodiments, the nanoparticles of the invention are administered intratumorally. In some embodiments, the nanoparticles of the invention are administered intracranially. In some embodiments, the nanoparticles of the invention are administered intraspinally. In some embodiments, methods of administering the nanoparticles of the invention results in delivery and accumulation of nanoparticles in a glioblastoma tumor. In some embodiments, systemic administration of the nanoparticles of the invention is followed by crossing of the blood brain barrier by the nanoparticles, and accumulation of the nanoparticles in a glioblastoma tumor.

In some embodiments, the nanoparticle agent conjugates of the invention are used in methods of delivering agents to a glioblastoma tumor. In some embodiments, the nanoparticle agent conjugates of the invention are administered systemically to a subject. In some embodiments, the nanoparticle agent conjugates of the invention are administered intratumorally. In some embodiments, the nanoparticle agent conjugates of the invention are administered intracranially. In some embodiments, the nanoparticle agent conjugates of the invention are administered intraspinally. In some embodiments, methods of administering the nanoparticle agent conjugates of the invention results in delivery and accumulation of nanoparticle agent conjugates in a glioblastoma tumor. In some embodiments, systemic administration of the nanoparticle agent conjugates of the invention is followed by crossing of the blood brain barrier by the nanoparticle agent conjugates, and accumulation of the nanoparticle agent conjugates in a glioblastoma tumor.

In some embodiments, the nanoparticle agent conjugates are used in methods of delivering a reporter to the glioblastoma tumor. In some embodiments, the nanoparticle agent conjugates are used in methods delivering an imaging agent to the glioblastoma tumor. In some embodiments the nanoparticle agent conjugates are used in methods of imaging a glioblastoma tumor. In some embodiments the nanoparticle agent conjugates are used in methods of detecting a glioblastoma tumor. In some embodiments the nanoparticle agent conjugates are used in methods of characterizing the size and location of a glioblastoma tumor. In some embodiments the nanoparticle agent conjugates are used in methods of detecting metastases from a glioblastoma tumor.

In some embodiments, the nanoparticle agent conjugates are used in methods of delivering to the glioblastoma tumor a mitotic inhibitor, an antitumor antibiotic, an immunomodulating agent, a gene therapy agent, an alkylating agent, an antiangiogenic agent, an antimetabolite, a chemoprotective agent, an antihormone agent, a corticosteroid, a photoactive therapeutic agent, an oligonucleotide, a radionuclide, a topoisomerase inhibitor, a tyrosine kinase inhibitor, or another agent. In some embodiments, the nanoparticle agent conjugates of the invention are used in methods for delivering doxorubicin to the glioblastoma tumor. In some embodiments, the nanoparticle agent conjugates of the invention are used in methods for delivering paclitaxel to the glioblastoma tumor. In some embodiments, the nanoparticle agent conjugates of the invention are used in methods for delivering an auristatin to the glioblastoma tumor.

In some embodiments, the nanoparticle agent conjugates of the invention are used in methods for treating a glioblastoma tumor with a mitotic inhibitor, an antitumor antibiotic, an immunomodulating agent, a gene therapy agent, an alkylating agent, an antiangiogenic agent, an antimetabolite, a chemoprotective agent, an antihormone agent, a corticosteroid, a photoactive therapeutic agent, an oligonucleotide, a radionuclide, a topoisomerase inhibitor, a tyrosine kinase inhibitor, or another agent. In some embodiments, the nanoparticle agent conjugates of the invention are used in methods for treating a glioblastoma tumor with doxorubicin. In some embodiments, the nanoparticle agent conjugates of the invention are used in methods for treating a glioblastoma tumor with paclitaxel. In some embodiments, the nanoparticle agent conjugates of the invention are used in methods for treating a glioblastoma tumor with an auristatin.

The inventions disclosed herein will be better understood from the experimental details which follow. However, one skilled in the art will readily appreciate that the specific methods and results discussed are merely illustrative of the inventions as described more fully in the claims which follow thereafter. Unless otherwise indicated, the disclosure is not limited to specific procedures, materials, or the like, as such may vary. It is also to be understood that the terminology used herein is for the purpose of describing particular embodiments only and is not intended to be limiting.

### EXAMPLES

### Example 1

### Delivery of Nanoparticles to a Glioblastoma Tumor

Three types of cylindrically shaped nanoparticles of 7-10 nanometers diameter and 50-150 nanometer length are used in this study. Gold nanowires are made with protein templating, genome-free protein nanophages or T4 tail fiber sections are made using techniques known in the art, and protein nano-tubes or chains using bottom up assembly techniques or DNA origami techniques. Nanoparticles disks of 17-20 nanometer diameter and 3-5 nanometer height are made. For example, gold nanodisks, protein nano-rings, genome-free viral capsid tobacco mosaic virus discs are used. The nanoparticles are characterized for statistical variance with TEM and SEM. Purification is accomplished using size exclusion chromatography. Penetration of glioblastoma tumors by nanoparticles is assessed using confocal microscopy for drug release, flow cytometry for intracellular uptake, synthetic membrane transport flow for endothelial gatekeeper cell transport, computational simulations and real-time intravital spectroscopy. Rod-shaped and disk-shaped nanoparticles will perform better than spherical symmetric shaped particles of similar diameter with several human glioblastoma cell lines including but not limited to U87, and with average tumor pore sizes that vary from 12 nanometers to 35 nanometers. Example 2

### Delivery of a Reporter to a Glioblastoma Tumor

Shape A1, gold nanoparticle disks with a diameter of 18 nanometers and a height of 5 nanometers are purchased from Cube Biotech. Shape A2, genome-free viral nanoparticle disks or two-layer stacked disk aggregates of Tobacco Mosaic Virus protein are made and then characterized structurally in the methods described in Diaz-Avalos et al, Biophysical Journal 74:1: 595-603 (1998) and Dedeo et al, Nanoletters 10:181-186 (2010), which is incorporated by reference in its entirety for all purposes. Shape B1, gold nanoparticle cylinders of diameter 10 nanometers and length of 50 nanometers are purchased from Sigma-Aldrich. Shape B2, cylindrically shaped protein nanoparticles are made from T4 viral tail fibers or from purified nanophages to produce nanoparticle cylinders with a diameter of 5 nanometers and a length of about 60 nanometers using the methods disclosed in Tim Harrah 2008, PhD Thesis Biomedical Engineering, Tufts University, Boston Mass., titled “Engineered bacteriophage T4 tail fiber proteins for nanotechnology,” and in Specthie et al. J. Mol. Biol. 228:720-724 (1992) both documents which are incorporated by reference in its entirety for all purposes. Shape C, gold nanoparticle spheres of diameter of 5 nanometers are purchased from Sigma Aldrich. All particles are characterized dimensionally and morphologically with TEM and SEM as described earlier.

The polymeric nanoparticle disks and cylinders are labeled with ⁶⁴Cu using a ⁶⁴Cu-DOTA conjugate (DOTA is 1,4,7,10-tetraazacyclododecane-N,N′,N″,N′″-tetraacetic acid) and PEGylated. The gold nanoparticles are labelled with gadolinium chelates to allow nan-invasive MRI imaging.

5-week old female athymic mice are implanted with luciferase labelled human glioblastoma tumor cells in the intracranial space IC, to produce intracerebral tumor xenografts. Size and growth of the intracranial tumor is measured twice weekly to time nanoparticle ⁶⁴Cu conjugate injection at the start of logarithmic growth for the glioblastoma tumors.

One set of the athymic mice with intracerebral tumor grafts are administered nanoparticle ⁶⁴Cu conjugates by tail injection at the start of logarithmic growth of the tumor xenografts. BLI and micro-PET is performed at the estimated peak or max uptake time of T=5 hours. After imaging and 5 hours after tail injection the mice are euthanized, various tissues are harvested, and the harvested tissues are tested for ⁶⁴Cu with scintillation count and IHC immunohistochemistry. Harvested tissues are glioblastoma tumors, contralateral healthy brain, heart, lungs, liver, intestines, kidney, spleen, pancreas, stomach, and blood.

A second set of athymic mice with intracerebral tumor grafts are administered gadolinium gold nanoparticle conjugates. This set of mice are monitored with MRI every 6 hours up to a 24 hour period. After this period, this set of mice is monitored twice daily and then euthanized at two weeks to monitor whether the gold and gadolinium conjugates are eliminated or retained in healthy tissue, particularly in the cardiac region.

The cylindrical and disc-shaped nanoparticle ⁶⁴Cu conjugates provide more accumulation in the glioblastoma tumors than the spherical nanoparticles. The gold cylindrical and disc-shaped nanoparticle gadolinium conjugates provide more accumulation in the glioblastoma tumors than the gold spherical nanoparticles. The gold, metallic, polymer, protein shapes of similar morphology and dimensions exhibit similar uptake rates. The shaped nanoparticle conjugates accumulate more ⁶⁴Cu in the tumor than the control ⁶⁴Cu free molecule alone. The gold nanoparticles show little retention in healthy tissue after two weeks.

### Example 3

### Speed of Shaped Nanoparticle Delivery to a Glioblastoma Tumor

Real-time intravital spectroscopy is used to determine the actual speed or rate of shaped nanoparticle delivery to an intracranial U87 glioblastoma tumor in an in vivo mouse model. Using the imaging methods described in Smith BR et al. Nano Lett., 2012, 12 (7), pp 3369-3377, which is incorporated by reference in its entirety for all purposes, fluorescent nanorods of dimension (10×50 nm), nanodiscs of dimension (5×17 nm), nanospheres of dimension (5 nm diameter) are tail-injected into athymic mice that have been prepared with intracranial human tumor injections as well as a specially designed intracranial imaging window or endoscope. Readings are taken at 1 hour periods starting with T=0 as the tail injection time. The in vivo speed or rate of the shaped nanoparticle delivery is compared with the other conventional imaging, in vitro and ex vivo bio-distribution and uptake techniques and data to improve calculation of controlled and time-release drug delivery, the curve and maximum tumor penetration and accumulation over time as well as better development of dosage regimes, toxicity and therapeutic window.

### Example 4

### Delivery of Doxorubicin to a Glioblastoma Tumor

Gold nanoparticle cylinders, protein nanophages and T4 viral tail fibers are produced with diameters of 5 nanometers and a lengths of about 60 nanometers using the methods disclosed in Tim Harrah 2008, PhD Thesis Biomedical Engineering, Tufts University, Boston Mass., titled “Engineered bacteriophage T4 tail fiber proteins for nanotechnology,” and Sadia Sattar, 2013 PhD Thesis Biochemistry, Massey University, NZ, titled “Filamentous phage-derived nanorods for applications in diagnostics and vaccines” which are all incorporated by reference in its entirety for all purposes. These three nanoparticle cylinders with the same dimension and shape but different material, fabrication and linker methods as described herein are conjugated with doxorubicin and appropriately PEGylated.

Dox is conjugated to the gold nanoparticles through an acid-labile hydrazone linker as described in Cheng, Y et al. Small 2014 Dec 29:10 (24), which is incorporated by reference in its entirety for all purposes.

5-week old female athymic mice are implanted with human glioblastoma tumor cells in the intracranial space IC, to produce intracerebral tumor xenografts. Size and growth of the intracranial tumor is measured twice weekly to time nanoparticle doxorubicin conjugate injection at the start of logarithmic growth for the glioblastoma tumors. The athymic mice with intracerebral tumor grafts are administered nanoparticle doxorubicin conjugates by tail injection at the start of logarithmic growth of the tumor xenografts.

At the peak tumor uptake time after tail injection (to be calculated as in Example 2 or 3) one set of the mice injected with protein nanoconjugates are imaged by BLI and PET, then euthanized, glioblastoma tumors are harvested, and the tumors are weighed with a scintillation counter as well as IHC immunohistology performed on all relevant organs.

A matching set of the mice injected with protein nanoconjugates are imaged twice a day for tumor growth and survival compared with the control groups.

At the peak tumor uptake time after tail injection, the mice injected with gold nanoconjugates are imaged by MRI for tumor size and growth. Several other imaging modalities for gold nanoparticles are described in Kircher et al. Nature Medicine, 2012, May (5), which is incorporated by reference in its entirety for all purposes.

The mice treated with nanoparticle doxorubicin conjugates show less tumor growth than untreated mice and mice treated with doxorubicin alone. The mice treated with nanoparticle doxorubicin conjugates show longer survival rates than untreated mice and mice treated with doxorubicin alone.

### Example 5

### Delivery of Doxorubicin to a Glioblastoma Tumor in Large Animal Models

Nanoparticle cylinders and discs with the aforementioned dimensions are produced using the methods disclosed above and are used to treat larger animals, specifically spontaneous glioblastoma in canine models. In the past, dogs with spontaneous glioblastoma have been treated successfully with local Convection-Enhanced Delivery (CED) administration of topoisomerase inhibitor nano-liposomal CPT-11 and canine spontaneous glioma has been shown to be a superior translational model system for new routes of administration, since spontaneous canine glioblastoma bears striking resemblances to human glioblastoma (Dickinson et al. Neuro Oncology. 2010 Sep: 12(9):928-940, which is incorporated by reference in its entirety for all purposes).

Doxorubicin drug-loaded nanoparticles of the dimensions and morphology described herein provides a nanoparticle treatment regime that can be systemically administered through intravenous injection, intra-nasal and intra-ocular routes of administration reaching difficult to access, deep or inoperable brain tumors and their fingerlike aggressive projections into healthy brain tissue.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

What is claimed is:

1. A method for delivering an agent to a glioblastoma tumor, comprising the steps of:
providing a nanoparticle wherein the nanoparticle is a cylinder having a diameter of about 5 to 20 nanometers and a length of about 10 to 100 nanometers, wherein the agent is conjugated to the nanoparticle; administering the nanoparticle agent conjugate to a subject wherein the glioblastoma tumor is located in a brain of the subject, and delivering the nanoparticle agent conjugate to the glioblastoma tumor.

2. The method of claim 1, wherein the nanoparticle is a viral nanoparticle.

3. The method of claim 1, wherein the nanoparticle further comprises a linker that conjugates the agent to the nanoparticle.

4. The method of claim 2, wherein the nanoparticle is a cylinder and has a diameter of 5 to 10 nanometers and a length of 25-50 nanometers.

5. The method of claim 3, wherein the linker is a cleavable linker, and further comprising the step of cleaving the linker to release the agent at the glioblastoma tumor.

6. The method of claim 5, wherein the linker comprises a peptide that can cleaved by a protease.

7. The method of claim 6, wherein the peptide comprises a valine linked to a citrulline by a peptide bond.

8. The method of claim 6, wherein the protease is a cathepsin B.

9. The method of claim 5, wherein the linker includes a self-immolative spacer.

10. The method of claim 1, wherein the agent is a reporter.

11. The method of claim 5, wherein the agent is selected from the group consisting of a mitotic inhibitor, an antitumor antibiotic, a plant alkaloid, an alkylating agent, an antimetabolite, and a radionuclide.

12. The method of claim 11, wherein the agent is a mitotic inhibitor.

13. The method of claim 11, wherein the agent is a plant alkaloid.

14. The method of claim 11, wherein the agent is an alkylating agent.

15. A method for delivering an agent to a glioblastoma tumor, comprising the steps of:
providing a viral nanoparticle wherein the viral nanoparticle is a cylinder having a diameter of about 5 to 20 nanometers and a length of about 10 to 100 nanometers, wherein the nanoparticle comprises a cleavable linker that conjugates the agent to the nanoparticle, administering the viral nanoparticle agent conjugate to a subject who has the glioblastoma tumor; and cleaving the linker to release the agent, whereby the viral nanoparticle agent conjugate delivers the agent to the glioblastoma tumor.

16. The method of claim 15, wherein the linker comprises a peptide that can cleaved by a protease.

17. The method of claim 16, wherein the protease is a cathepsin B.

18. The method of claim 15, wherein the agent is a reporter.

19. The method of claim 5, wherein the agent is selected from the group consisting of a mitotic inhibitor, an antitumor antibiotic, a plant alkaloid, an alkylating agent, an antimetabolite, and a radionuclide.

20. The method of claim 19, wherein the agent is an alkylating agent.
